(19) United States
(12) Patent Application Publication
(10) Pub. No.: US 2019/0154785 A1
(43) Pub. Date: May 23, 2019

(54) Cardiac and Respiratory Self-Gated Motion-Corrected Free-Breathing Spiral Cine Imaging

(71) Applicant: University of Virginia Patent Foundation, Charlottesville, VA (US)

(72) Inventors: Ruixi Zhou, Charlottesville, VA (US); Yang Yang, Charlottesville, VA (US); Michael Salerno, Charlottesville, VA (US)

(21) Appl. No.: 16/195,424

(22) Filed: Nov. 19, 2018

Related U.S. Application Data
(60) Provisional application No. 62/743,588, filed on Oct. 10, 2018.
(60) Provisional application No. 62/587,773, filed on Nov. 17, 2017.

(51) Int. Cl. G01R 33/563; G01R 33/567; A61B 5/055

(52) U.S. Cl. CPC G01R 33/56325; G01R 33/5676; G01R 33/56509; G01R 33/56316; A61B 5/055

(57) ABSTRACT

In some aspects, the present disclosure relates to free-breathing cine imaging of an area of interest of a subject. In one embodiment, a method includes acquiring, during free breathing of the subject, magnetic resonance imaging data corresponding to an area of interest of a subject that comprises the heart, wherein the acquiring comprises applying a pulse sequence with a spiral trajectory. The method also includes performing cardiac self-gating using a self-gating signal extracted from a central region of k-space, and performing respiratory motion correction to compensate for changes in the heart position during respiratory motion, wherein the motion correction comprises rigid or non-rigid registration to determine corrective displacements. The method also includes performing image reconstruction to produce cine images of the area of interest over a plurality of heart-beats.

### CROSS-REFERENCE TO RELATED APPLICATIONS

This Application claims priority to, and benefit under 35 U.S.C. § 119(e) of, U.S. Provisional Patent Application No. 62/587,773, filed Nov. 17, 2017, and U.S. Provisional Patent Application No. 62/743,588, filed Oct. 10, 2018, both of which applications are hereby incorporated by reference herein in their entireties as if fully set forth below.

### STATEMENT REGARDING GOVERNMENT SUPPORT

This invention was made with government support under Grant Nos. HL112910 and HL131919, awarded by the National Institutes of Health. The government has certain rights in this invention.

### BACKGROUND

Cardiac magnetic resonance (CMR) cine imaging is widely regarded as the gold-standard technique for the non-invasive assessment of cardiac function. Typically, images are acquired using a breath-held 2D segmented electrocardiography-gated (ECG-gated) balanced steady-state free precession (bSSFP) pulse sequence. Prospective or retrospective ECG-gating is used to synchronize segmented data acquisition to the cardiac cycle over multiple heartbeats during a breath-hold to generate images at multiple cardiac phases across the cardiac cycle.

The approach of using electrocardiography (ECG) triggering and breath-hold acquisition has several limitations. Firstly, the ECG signal can be distorted due to the magnetohydrodynamic effect [1], rapid switch of magnetic gradients [2], as well as radiofrequency interference [3,4] resulting in mis-triggering. This distortion is worse at higher field strengths such as 3 T. Furthermore, placement of the ECG leads requires expertise and increases the time to prepare the patient for the CMR exam. Additionally, a significant number of patients are not able to adequately hold their breath during cine acquisition, resulting in motion artifacts and the need to repeat image acquisition of the same slice location on subsequent breath-holds. Even if the patient can perform good breath-holds, this approach is inefficient, as it requires 10-12 breath-holds to cover the left ventricle (LV) and requires coordination between the operator and the patient.

To partially alleviate issues caused by cardiac and respiratory motion, the conventional solutions can be separated into 3 categories: navigator-based methods, real-time methods, and self-gating methods. Navigator-echo based methods which accept or reject data based on the position of the diaphragm have been used to account for respiratory motion [5-8]. However, the usage of diaphragmatic navigators typically precludes retrospective ECG gating, and the total scanning time is prolonged depending on respiratory gating efficiency. More recently, projection navigators acquired during steady state free precession (SSFP) have been utilized to perform respiratory tracking without the need for a separate diaphragmatic navigator [9], but this approach is still limited by navigator gated efficiency. These methods may account for respiratory motion, but they do not provide a mechanism to account for cardiac motion.

Other “self-navigated” techniques [10,11] have been proposed to eliminate the need for ECG synchronization by acquiring and processing additional magnetic resonance (MR) signals to derive cardiac cycle timing information. Relative to ECG-gated techniques, methods that use extra lines of data to acquire the self-gating signals result in decreased imaging efficiency. In the clinical setting, when the ECG or breath-holds do not perform adequately, operators may resort to real-time imaging techniques. Although these real-time methods do not require ECG gating, they have been shown to sacrifice spatial and/or temporal resolution [12-14].

Thus, there is a growing interest in self-gated free-breathing approaches that do not compromise achievable resolution. While applying ECG triggering, some studies [15-20] rely on the acquired data itself to derive respiratory signals, such as using filtered data or projection data. The acquired dataset is usually separated into different respiratory states [18,21,22] and reconstruction is performed using data from a subset of the respiratory states, or by using motion correction to combine data from different respiratory states. Other studies extract the cardiac motion from the acquired data during breath-holds [23-25] or free-breathing [21,22,26]. Previously reported cardiac self-gating approaches have been shown to use the k-space center point [11,27] or center k-space line [12,15,16,23] as navigator signals. Some techniques [23,28] also used image-based methods to obtain self-gating signals.

In terms of sampling strategy, most studies have utilized Cartesian and radial trajectories [10,11,15-17,23,28]. A recent study has used a breath-held cardiac self-gated spiral technique to quantify coronary artery vasodilation [25]. The use of free-breathing cardiac and respiratory self-gated golden angle spiral trajectories for the evaluation of cardiac anatomy and function has not been explored to date.

There are still several limitations to be overcome. Firstly, these above-described approaches usually require careful selection of receive coil elements to obtain self-gating signals, as each coil has different sensitivity to cardiac motion and respiratory motion. Secondly, it is inefficient to discard data acquired at undesired respiratory phases. While imaging at 1.5 T, SSFP sequences are more efficient and have better contrast to noise; for imaging at 3 T, balanced steady state free precession sequences typically require frequency-scouts and careful shimming to avoid off-resonance artifacts such as banding artifact and may be less robust for automatic free breathing acquisition at field strengths at or above 3 T.

It is with respect to these and other considerations that the various aspects of the present disclosure as described below are presented.

### SUMMARY

In some aspects, the present disclosure relates to systems, methods, and computer-readable media for cardiac and respiratory self-gated motion-corrected free-breathing spiral cine imaging. In one aspect, the present disclosure relates to a method for free-breathing cine imaging of an area of interest of a subject. In one embodiment, the method includes acquiring, during free breathing of the subject, magnetic resonance imaging data corresponding to an area of interest of a subject that comprises the heart, wherein the acquiring comprises applying a pulse sequence with a spiral trajectory. The method also includes performing cardiac self-gating using a self-gating signal extracted from a central region of k-space, and performing respiratory motion correction to compensate for changes in the heart position during respiratory motion, wherein the motion correction comprises rigid or non-rigid registration to determine corrective displacements. The method also includes performing image reconstruction to produce cine images of the area of interest over a plurality of heart-beats.

In another aspect, the present disclosure relates to a system for free-breathing cine imaging of an area of interest of a subject. In one embodiment, the system includes a data acquisition device configured to acquire, during free breathing of the subject, magnetic resonance imaging data corresponding to an area of interest of a subject that comprises the heart, wherein the acquiring comprises applying a pulse sequence with a spiral trajectory. The system also includes one or more processors coupled to the data acquisition device and configured to cause the system to perform specific functions that include: performing cardiac self-gating using a self-gating signal extracted from a central region of k-space; performing respiratory motion correction to compensate for changes in the heart position during respiratory motion, wherein the motion correction comprises rigid or non-rigid registration to determine corrective displacements; and performing image reconstruction to produce cine images of the area of interest over a plurality of heart-beats.

In another aspect, the present disclosure relates to a non-transitory computer-readable medium having stored instructions that, when executed by one or more processors, cause a magnetic resonance imaging system to perform specific functions that include: acquiring, during free breathing of the subject, magnetic resonance imaging data corresponding to an area of interest of a subject that comprises the heart, wherein the acquiring comprises applying a pulse sequence with a spiral trajectory; performing cardiac self-gating using a self-gating signal extracted from a central region of k-space; performing respiratory motion correction to compensate for changes in the heart position during respiratory motion, wherein the motion correction comprises rigid or non-rigid registration to determine corrective displacements; and performing image reconstruction to produce cine images of the area of interest over a plurality of heart-beats.

Some embodiments of the present disclosure relate to a free-breathing continuous-acquisition respiratory and cardiac self-gated golden angle spiral cine pulse technique, which is sometimes referred to herein as SPiral Acquisition with Respiratory and Cardiac Self-Gating (SPARCS). In one example implementation of the present disclosure, data was acquired using a spiral interleaf rotated by the golden-angle (137.51°) in time. The cardiac self-gating signal was extracted using principal component analysis (PCA) on a gridded 8×8 central region of k-space for each spiral, and the respiratory motion was derived from rigid registration for each heartbeat. Images were reconstructed with a rigid-motion compensated low rank and sparse (L+S) technique [29]. Free-breathing self-gated spiral cine imaging in accordance with some embodiments of the present disclosure demonstrated high image quality providing whole heart coverage with clinical spatial resolution (1.25 mm×1.25 mm) and temporal resolution (<40 ms) in under 3 minutes. Other embodiments of the present disclosure relate to extending aspects of SPARCS according to certain aspects and embodiments disclosed herein to simultaneous multi-slice (SMS) imaging and 3D acquisition techniques.

Some embodiments of the present disclosure relate to extending aspects of SPARCS to obtain other information in addition to cine imaging, such as so-called T1 mapping, sometimes referred to herein as Cine and T1-SPARCS (CAT-SPARCS). In some embodiments, CAT-SPARCS provides for the acquisition of parametric images of myocardial T1 relaxation times in addition to the cine images. When applied following the application of a contrast agent, CAT-SPARCS according to some embodiments can simultaneously acquire information for self-gated cine imaging, self-gated T1 mapping, and late gadolinium enhancement (LGE) imaging to visualize myocardial scar. CAT-SPARCS can also be extended with other magnetization preparation pulses or other modifications to image other properties such as T2 or magnetization transfer.

Other aspects and features according to the example embodiments of the present disclosure will become apparent to those of ordinary skill in the art, upon reviewing the following detailed description in conjunction with the accompanying figures.

### BRIEF DESCRIPTION OF THE DRAWINGS

The patent or application file contains at least one drawing executed in color. Copies of this patent or patent application publication with the color drawing(s) will be provided by the Office upon request and payment of the necessary fee. Reference will now be made to the accompanying drawings, which are not necessarily drawn to scale.

FIG. 1 shows a flow diagram illustrating operations of a Spiral Acquisition with Respiratory and Cardiac Self-Gating (SPARCS) technique in accordance with one embodiment of the present disclosure, wherein: (a) shows gridding of an 8×8 fully sampled central region of k-space for each spiral arm for all receiver coils through time; (b) shows principal component analysis (PCA) performed on this data to derive temporal basis functions; and (c) shows frequency spectrum analysis used to find the cardiac motion-related component. The cardiac motion component extracted by peak detection and use of a threshold to exclude potential artefactual peaks are shown in (d) and (e) of FIG. 1, and (f) shows combining the same cardiac phase in different R-R intervals to obtain a high-quality image series containing the cardiac motion. Automatic heart detection is shown in (g), and (h) shows rigid registration performed over the heart region of interest (ROI). Automatic coil selection is shown at (i), and (j) shows the extracted respiratory motion component. Retrospective binning is shown in (k), and (l) shows image reconstruction using low rank and sparse decomposition.

FIG. 2 shows cardiac gating efficiency, wherein a Bland-Altman plot (a) indicates a non-significant bias of 0.9054 ms of R-R interval length across the subjects, and (b) shows a positive correlation relationship (R²=0.96) between the ECG signal and the extracted cardiac trigger.

FIG. 3 shows motion correction performance of one subject, wherein (a) shows the rigid registration displacement in x (left-right) and y (head-foot) direction, and (c) and (d) exhibit the x-t profile, in which the position is indicated in (b), before and after registration.

FIG. 4 shows automatic coil selection demonstrated in one subject, wherein: (a) shows the coil images over the heart sorted by Eq (1); and (b) and (c) show the reconstructed images before and after the proposed coil selection method. Red arrows point out the aliasing caused by remote coils.

FIG. 5 shows single slice subject reconstruction results, wherein (a), (b), and (c) represent three subjects. The first rows of all subjects correspond to reconstructed images using 16 s data, and the second rows are the images using 8 s data. The first two columns show the systole and diastole images using a non-uniform fast Fourier transform (NUFFT), and the next two columns use L+S. The last two columns show the x-t profiles on NUFFT and L+S results using 16 s and 8 s data. Red arrows point out one improvement of aliasing for this specific combination of spiral k-space trajectories and reconstruction techniques

FIG. 6 shows blind grades for all subjects for a particular embodiment of pulse sequence and image reconstruction method. The bar plot shows the score using 16 s NUFFT, 16 s N+S, 8 s NUFFT, and 8 s L+S graded by two blinded cardiologists; “*” indicates significant difference (p<0.001).

FIG. 7 shows one subject of whole heart coverage reconstruction results. The top two rows of the images are L+S diastole frames across all slices using 16 s data (a) and 8 s data (b), and the bottom two rows are L+S systole frames shown with both 16 s (c) and 8 s data (d).

FIG. 8 shows Bland-Altman plots of ejection fraction (EF) for ten whole heart coverage subjects, wherein (a) shows a Bland-Altman plot of EF calculated from 16 s L+S SPARCS image results and Cartesian SSFP image results, and (b) shows a Bland-Altman plot of EF calculated from 8 s L+S SPARCS image results and Cartesian SSFP image results.

FIG. 9 shows example cine SMS (a) and 3D SPARCS (b) demonstrating extending SPARCS to cover multiple slices simultaneously or image the whole heart in one acquisition, according to some embodiments of the present disclosure. In the embodiment shown in FIG. 9(a), 3 slices are simultaneously excited with phase modulation following a Hadamard encoding scheme. Images were reconstructed using SMS-L1 SPIRIT [45]. SMS at rates 2-4 are feasible. FIG. 9(b) shows example images from a 3D SPARCS acquisition. In this embodiment the same trajectory is used for each of the 10 partitions so that the data can be Fourier transformed in the through-slice direction separating the reconstruction into 10 2D reconstructions, which can be performed using an L+S approach. The 3D approach can enable whole heart coverage and the ability to perform respiratory motion correction in the through-plane direction, which can be difficult for the 2D acquisition. When images are acquired post-contrast, there is improved contrast between the LV cavity and the myocardium The use of SSFP acquisition can also improve the contrast between the blood pool and the cavity. The trajectory can be modified in the partition direction to further spread aliasing energy, at the expense of the need for a fully 3D volumetric image reconstruction.

FIG. 10 shows aspects of extending SPARCS in accordance with some embodiments to use with SMS and 3D acquisitions. The upper-left image of FIG. 10 shows 3D data acquired with slab excitation, and the upper-right side plot of FIG. 10 shows the respiratory signal (blue line) and cardiac signal (orange line) derived using PCA. The lower-right side plots of FIG. 10 shows that, by filtering, the extracted ECG component can be derived, and shows good correlation with actual ECG, demonstrating that the signal can be used for self-gating.

FIG. 11 shows aspects of extending SPARCS in accordance with some embodiments using magnetization preparation to simultaneously acquire cine, LGE, and T1 maps. This technique can be referred to herein as “CAT-SPARCS”. In one implementation, every 2-8 seconds during the continuous acquisition, an inversion pulse is applied. This results in the signal intensity recovering along a T1* curve, from which the myocardial T1 can be determined. The data can be separated into a portion which is sensitive to the T1 recovery of the magnetization (denoted as “T1 mapping data”) and a steady-state signal component wherein cine images of cardiac function can be obtained. Data over multiple cardiac cycles can be combined as in SPARCS to improve signal to noise and fitting accuracy for the T1 maps. If the technique is performed following contrast administration, an image can be created at a fixed time relative to the inversion pulse at a time where the normal myocardial signal is at its “null” point to create a late gadolinium enhanced image to assess myocardial scar.

As the signal intensity is changing during SPARCS acquisition, when the central 8×8 pixel matrix is gridded, signal phase correction can be applied prior to PCA analysis, to obtain a temporal basis function which is sensitive to the T1 recovery. The other two temporal basis functions can be used for respiratory motion correction and for cardiac self-gating as in the SPARCS technique. A similar approach to SPARCS can be used for heart detection, and automatic coil selection.

FIG. 12 shows a flow diagram illustrating operations for extracting the cardiac, respiratory, and signal intensity change caused by inversion recovery components for self-gating, according to some embodiments of the present disclosure, wherein (a) shows gridding 8×8 center k-space across all coils through time, (b) shows PCA, (c) shows cardiac component filtering and peak detection, compared with recorded ECG signal, (d) shows retrospective binning, (e) shows automatic heart detection, and (f) shows automatic coil detection.

FIG. 13 shows registered data separated into cine, LGE and T1 based on extracted intensity change component (a). The dashed red line in (a) represents the threshold used to separate the cine and LGE portions. After retrospective binning (b), the cine data (c) is derived from the flat “steady-state portion” (shown as the region between the red lines in (a)) of the signal recovery following the inversion pulse. The rest of the curve which is T1* weighted is used for LGE and T1 mapping. FIG. 13(e) shows a sliding window to determine TI. By choosing an image where the normal myocardium is nulled “black” shown as the green-circle (in (e)), an LGE image can be obtained (d). By reconstructing images at different times following the inversion pulse the T1* recovery can be fit to a model, which enables creation of a T1 map (f).

FIG. 14 shows example reconstructed cine images at systole and diastole as well as LGE images at three slice locations obtained using CAT-SPARCS according to some embodiments of the present disclosure.

FIG. 15 is a system diagram illustrating an operating environment capable of implementing aspects of the present disclosure in accordance with one or more embodiments.

FIG. 16 is a computer architecture diagram showing a computing system capable of implementing aspects of the present disclosure in accordance with one or more embodiments.

### DETAILED DESCRIPTION

In some aspects, the present disclosure relates to systems, methods, and computer-readable media for cardiac and respiratory self-gated motion-corrected free-breathing spiral cine imaging.

Although example embodiments of the present disclosure are explained in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an” and “the” include plural referents unless the context clearly dictates otherwise.

Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest” (ROI).

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. For example, [3] refers to the 3^{rd} reference in the list, namely Shetty A N. Suppression of Radiofrequency Interference in Cardiac Gated MRI: A Simple Design. Magn. Reson. Med. 1988; 8:84-8. All references cited and discussed in this specification are incorporated herein by reference in their entireties and to the same extent as if each reference was individually incorporated by reference.

A detailed description of aspects of the present disclosure, in accordance with various example embodiments, will now be provided with reference to the accompanying drawings. The drawings form a part hereof and show, by way of illustration, specific embodiments and examples. In referring to the drawings, like numerals represent like elements throughout the several figures.

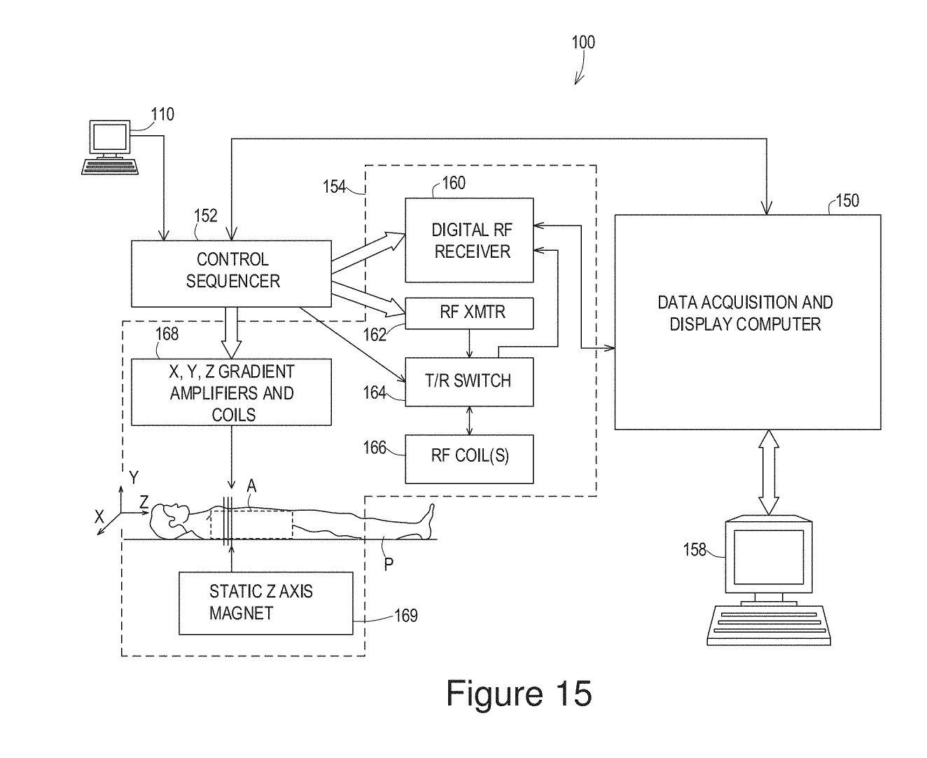

FIG. 15 is a system diagram illustrating an operating environment capable of implementing aspects of the present disclosure in accordance with one or more example embodiments. FIG. 15 illustrates an example of a magnetic resonance imaging (MRI) system 100, including a data acquisition and display computer 150 coupled to an operator console 110, an MRI real-time control sequencer 152, and an MRI subsystem 154. The MM subsystem 154 may include XYZ magnetic gradient coils and associated amplifiers 168, a static Z-axis magnet 169, a digital RF transmitter 162, a digital RF receiver 160, a transmit/receive switch 164, and RF coil(s) 166. The MM subsystem 154 may be controlled in real time by control sequencer 152 to generate magnetic and radio frequency fields that stimulate magnetic resonance phenomena in a subject P to be imaged, for example, to implement magnetic resonance imaging sequences in accordance with various example embodiments of the present disclosure described herein. An image of an area of interest A of the subject P (which may also be referred to herein as a “region of interest”) may be shown on display 158. The display 158 may be implemented through a variety of output interfaces, including a monitor, printer, or data storage.

The area of interest A corresponds to a region associated with one or more physiological activities in subject P. The area of interest shown in the example embodiment of FIG. 15 corresponds to a chest region of subject P, but it should be appreciated that the area of interest for purposes of implementing various aspects of the disclosure presented herein is not limited to the chest area. It should be recognized and appreciated that the area of interest in various embodiments may encompass various areas of subject P associated with various physiological characteristics, such as, but not limited to the heart region. Physiological activities that may be evaluated by methods and systems in accordance with various embodiments of the present disclosure may include but are not limited to cardiac activity and conditions.

It should be appreciated that any number and type of computer-based medical imaging systems or components, including various types of commercially available medical imaging systems and components, may be used to practice certain aspects of the present disclosure. Systems as described herein with respect to example embodiments are not intended to be specifically limited to magnetic resonance imaging (MRI) implementations or the particular system shown in FIG. 15.

One or more data acquisition or data collection steps as described herein in accordance with one or more embodiments may include acquiring, collecting, receiving, or otherwise obtaining data such as imaging data corresponding to an area of interest. By way of example, data acquisition or collection may include acquiring data via a data acquisition device, receiving data from an on-site or off-site data acquisition device or from another data collection, storage, or processing device. Similarly, data acquisition or data collection devices of a system in accordance with one or more embodiments of the present disclosure may include any device configured to acquire, collect, or otherwise obtain data, or to receive data from a data acquisition device within the system, an independent data acquisition device located on-site or off-site, or another data collection, storage, or processing device.

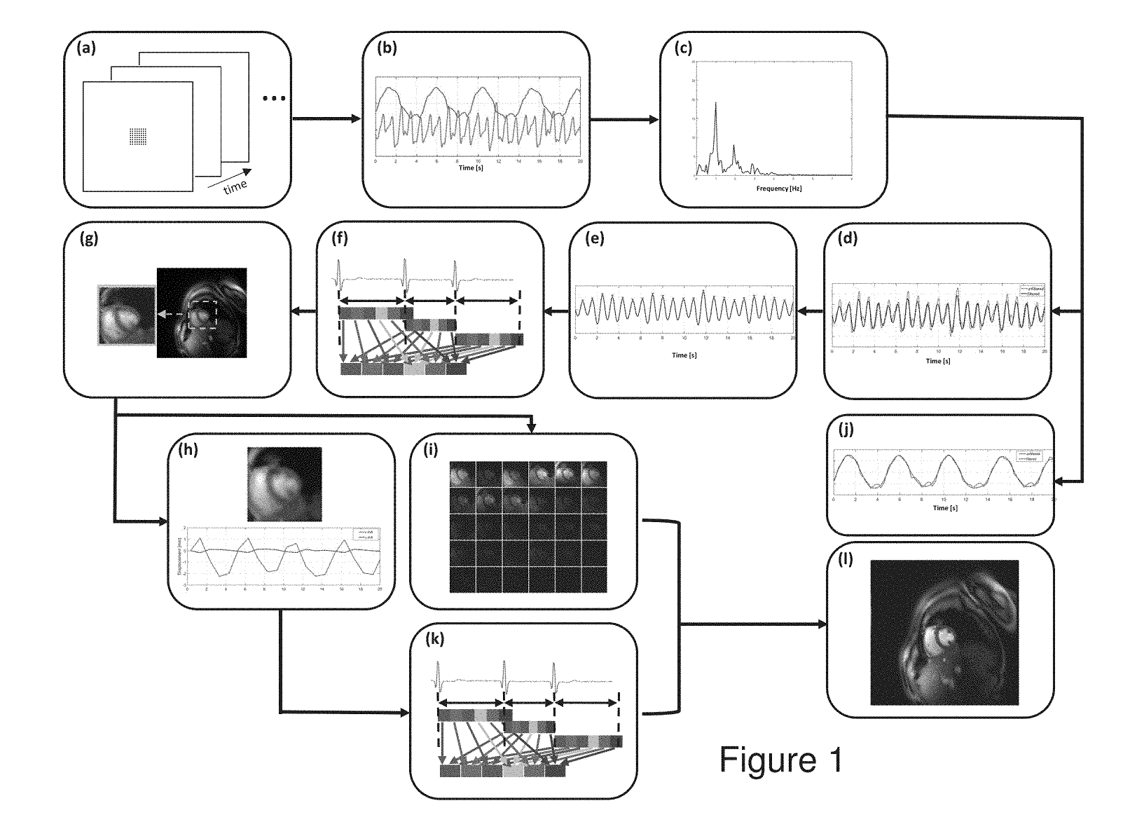

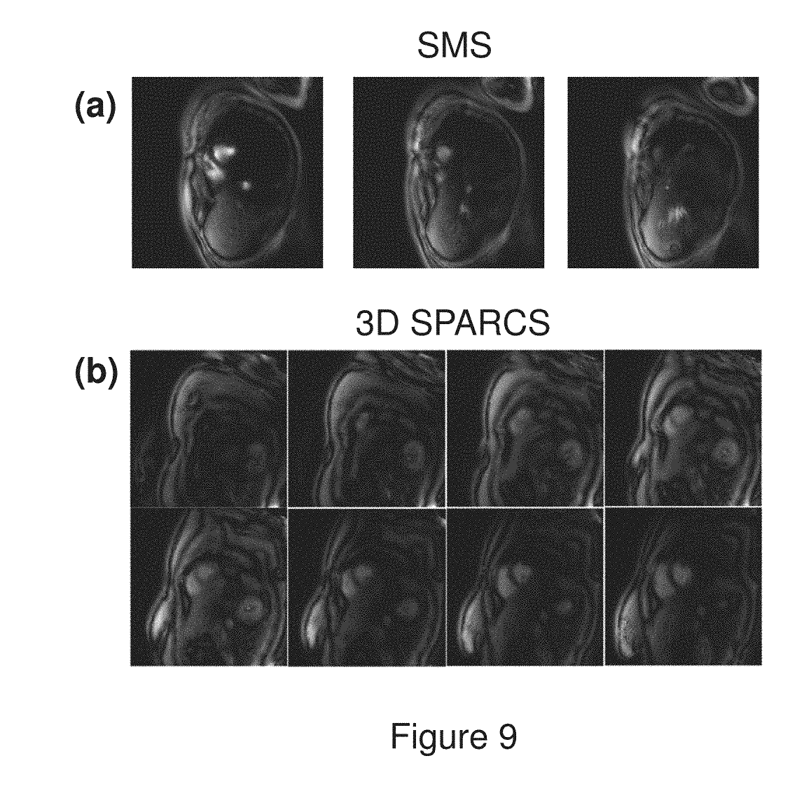

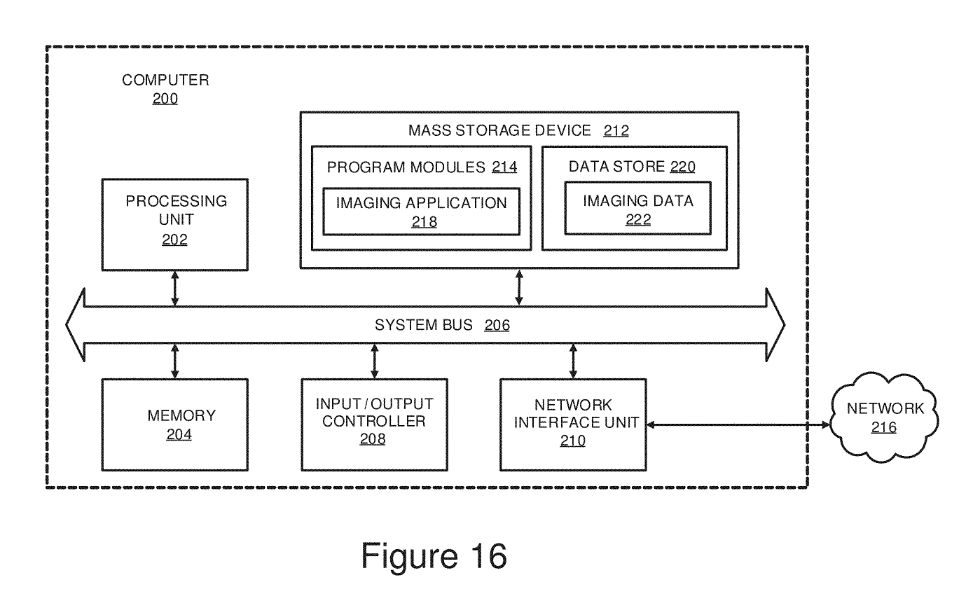

FIG. 16 is a computer architecture diagram showing a computing system capable of implementing aspects of the present disclosure in accordance with one or more embodiments described herein. A computer 200 may be configured to perform one or more functions associated with embodiments illustrated in one or more of FIGS. 1-15. For example, the computer 200 may be configured to perform various aspects shown in FIG. 1 and described below. It should be appreciated that the computer 200 may be implemented within a single computing device or a computing system formed with multiple connected computing devices. The computer 200 may be configured to perform various distributed computing tasks, in which processing and/or storage resources may be distributed among the multiple devices. The data acquisition and display computer 150 and/or operator console 110 of the system shown in FIG. 15 may include one or more systems and components of the computer 200.

As shown, the computer 200 includes a processing unit 202 (“CPU”), a system memory 204, and a system bus 206 that couples the memory 204 to the CPU 202. The computer 200 further includes a mass storage device 212 for storing program modules 214. The program modules 214 may be operable to perform associated with embodiments illustrated in one or more of FIGS. 1-15 discussed herein. The program modules 214 may include an imaging application 218 for performing data acquisition and/or processing functions as described herein, for example to acquire and/or process image data corresponding to magnetic resonance imaging of an area of interest. The computer 200 can include a data store 220 for storing data that may include imaging-related data 222 such as acquired data from the implementation of magnetic resonance imaging in accordance with various embodiments of the present disclosure.

The mass storage device 212 is connected to the CPU 202 through a mass storage controller (not shown) connected to the bus 206. The mass storage device 212 and its associated computer-storage media provide non-volatile storage for the computer 200. Although the description of computer-storage media contained herein refers to a mass storage device, such as a hard disk or CD-ROM drive, it should be appreciated by those skilled in the art that computer-storage media can be any available computer storage media that can be accessed by the computer 200.

By way of example and not limitation, computer storage media (also referred to herein as “computer-readable storage medium” or “computer-readable storage media”) may include volatile and non-volatile, removable and non-removable media implemented in any method or technology for storage of information such as computer-storage instructions, data structures, program modules, or other data. For example, computer storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer 200. “Computer storage media”, “computer-readable storage medium” or “computer-readable storage media” as described herein do not include transitory signals.

According to various embodiments, the computer 200 may operate in a networked environment using connections to other local or remote computers through a network 216 via a network interface unit 210 connected to the bus 206. The network interface unit 210 may facilitate connection of the computing device inputs and outputs to one or more suitable networks and/or connections such as a local area network (LAN), a wide area network (WAN), the Internet, a cellular network, a radio frequency (RF) network, a Bluetooth-enabled network, a Wi-Fi enabled network, a satellite-based network, or other wired and/or wireless networks for communication with external devices and/or systems. The computer 200 may also include an input/output controller 208 for receiving and processing input from any of a number of input devices. Input devices may include one or more of keyboards, mice, stylus, touchscreens, microphones, audio capturing devices, and image/video capturing devices. An end user may utilize the input devices to interact with a user interface, for example a graphical user interface, for managing various functions performed by the computer 200. The bus 206 may enable the processing unit 202 to read code and/or data to/from the mass storage device 212 or other computer-storage media. The computer-storage media may represent apparatus in the form of storage elements that are implemented using any suitable technology, including but not limited to semiconductors, magnetic materials, optics, or the like. The computer-storage media may represent memory components, whether characterized as RAM, ROM, flash, or other types of technology.

The computer storage media may also represent secondary storage, whether implemented as hard drives or otherwise. Hard drive implementations may be characterized as solid state, or may include rotating media storing magnetically-encoded information. The program modules 214, which include the imaging application 218, may include instructions that, when loaded into the processing unit 202 and executed, cause the computer 200 to provide functions associated with one or more example embodiments and implementations illustrated in FIGS. 1-15. The program modules 214 may also provide various tools or techniques by which the computer 200 may participate within the overall systems or operating environments using the components, flows, and data structures discussed throughout this description.

In general, the program modules 214 may, when loaded into the processing unit 202 and executed, transform the processing unit 202 and the overall computer 200 from a general-purpose computing system into a special-purpose computing system. The processing unit 202 may be constructed from any number of transistors or other discrete circuit elements, which may individually or collectively assume any number of states. More specifically, the processing unit 202 may operate as a finite-state machine, in response to executable instructions contained within the program modules 214. These computer-executable instructions may transform the processing unit 202 by specifying how the processing unit 202 transitions between states, thereby transforming the transistors or other discrete hardware elements constituting the processing unit 202. Encoding the program modules 214 may also transform the physical structure of the computer-storage media. The specific transformation of physical structure may depend on various factors, in different implementations of this description. Examples of such factors may include, but are not limited to the technology used to implement the computer-storage media, whether the computer storage media are characterized as primary or secondary storage, and the like. For example, if the computer storage media are implemented as semiconductor-based memory, the program modules 214 may transform the physical state of the semiconductor memory, when the software is encoded therein. For example, the program modules 214 may transform the state of transistors, capacitors, or other discrete circuit elements constituting the semiconductor memory.

As another example, the computer storage media may be implemented using magnetic or optical technology. In such implementations, the program modules 214 may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present description, with the foregoing examples provided only to facilitate this discussion.

Further details of certain example embodiments of the present disclosure will now be discussed.

As mentioned in some detail above in the “Background” section of the present Application, in current clinical practice, breath-held ECG-gated Cartesian cine images are typically acquired to assess cardiac function. This approach can be inefficient, as it can require 10-12 breath-holds to cover the left ventricle, and can be susceptible to both respiratory-motion and electrocardiography (ECG) gating artifacts, particularly at 3 T. To address these limitations, some embodiments of the present disclosure provide a continuous-acquisition respiratory and cardiac self-gated spiral sequence and motion-compensated reconstruction strategy for free breathing cine imaging, which may also be referred to herein as “Spiral Acquisition with Respiratory and Cardiac Self-Gating” or “SPARCS”.

In accordance with an example implementation according to the present disclosure, and as will be described further below, cine data was acquired continuously on a 3 T scanner over 8-16 seconds without ECG gating or breath-holding using a golden-angle (137.51°) rotated gradient echo spiral pulse sequence with one interleaf per excitation. Cardiac motion information was extracted by applying principal component analysis (PCA) on the gridded 8×8 center k-space. Respiratory motion was corrected by rigid registration on each heartbeat. Images were reconstructed using a low rank and sparse (L+S) technique. This strategy was evaluated in 33 healthy subjects and 5 subjects undergoing clinical CMR studies. Image quality was scored in a blinded fashion by two experienced cardiologists using a 5-point score (1: poor-5: excellent). In 10 subjects with whole heart coverage, left ventricular ejection fraction (LVEF) from the spiral technique was compared to a standard ECG gated steady state free precession (SSFP) breath-hold cine sequence. In this example implementation, the self-gated signal could be extracted in all cases and demonstrated close agreement with the acquired ECG signal (mean bias 0.9 ms). The mean image scores across all subjects were 4.02 and 4.22 for the images reconstructed using L+S with 8 or 16 second data acquisition (p>0.1). There was good agreement between the ejection fraction derived from the SPARCS sequence and the gold-standard cine SSFP technique.

As demonstrated by the example implementation described above and covered in further detail below, the SPARCS technique according to some embodiments of the present disclosure can successfully image cardiac function without the need for ECG gating or breath-holding. With an 8-second data acquisition per slice, whole heart cine images with clinically acceptable spatial and temporal resolution and image quality can be acquired in less than 90 seconds of free-breathing acquisition, which significantly improves the efficiency of cardiac cine imaging.

Other embodiments of the present disclosure relate to extending aspects of SPARCS according to certain aspects and embodiments disclosed herein to simultaneous multi-slice (SMS) imaging and 3D acquisition techniques. Self-gating can be performed either with SMS imaging, or by using 3D volumetric excitation techniques.

Spiral SMS acquisition enables multiple slices to be imaged simultaneously, increasing efficiency without increasing acquisition time. It has been demonstrated that multi-band factors of 2-4 are typically feasible. This enables data collection of 12 slices in <3 minutes with a 30 second acquisition time per set of slices. Alternatively, 3D stack-of-spirals acquisition is an efficient trajectory to collect volumetric data.

Spiral-cine images can be successfully acquired from 3 slice locations simultaneously. Data is collected using a golden-angle spiral trajectory with Hadamard encoding, so calibration data for each slice position is obtained automatically without need for additional data acquisition. This technique uses a straight-forward extension of SMS and SPARCS techniques and can improve efficiency 3 fold, allowing for more data to be acquired for each slice location without prolonging the time needed to image 3 slices. In some implementations, all of the data can be acquired in a single 3D acquisition. The self-gated signal can be derived from the 3D-excited volume. Also, 3-directional navigators can be imbedded and used to aid for respiratory compensation.

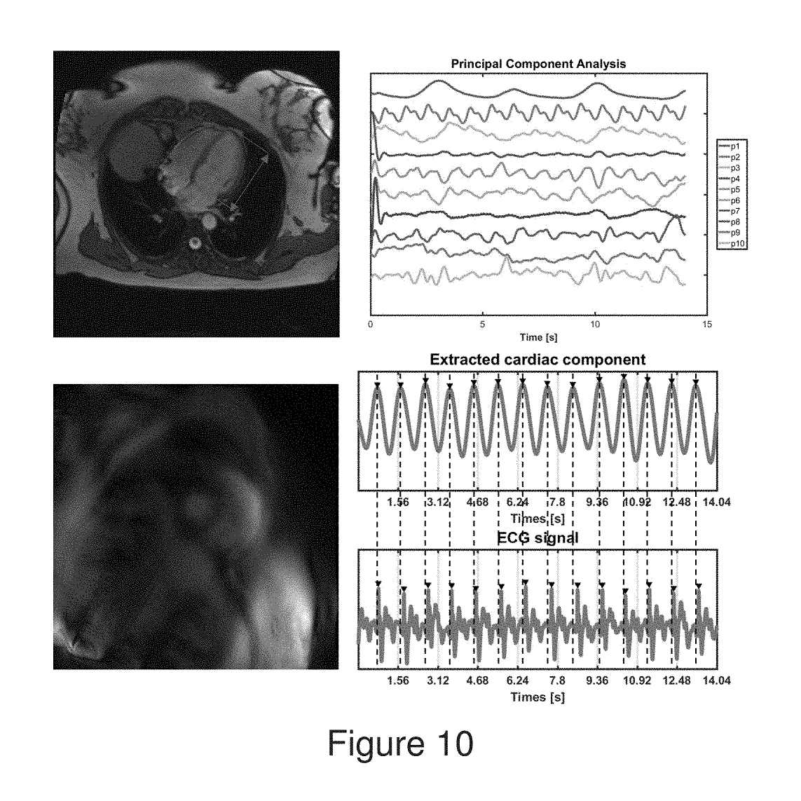

Now referring specifically to FIG. 10, 3D data is acquired with slab excitation. Using PCA (upper right image), the respiratory signal (blue line) and cardiac signal (orange line) can be derived. The illustrated data shows good correlation with the actual ECG, demonstrating that the signal can be used for self-gating.

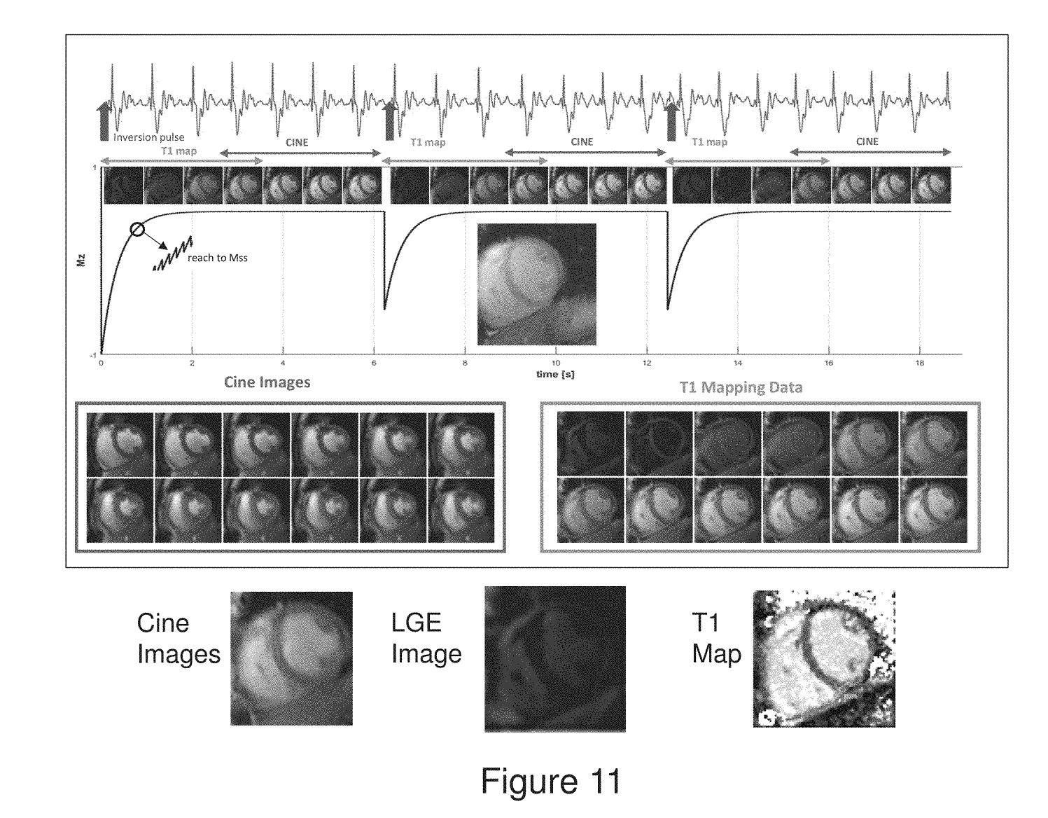

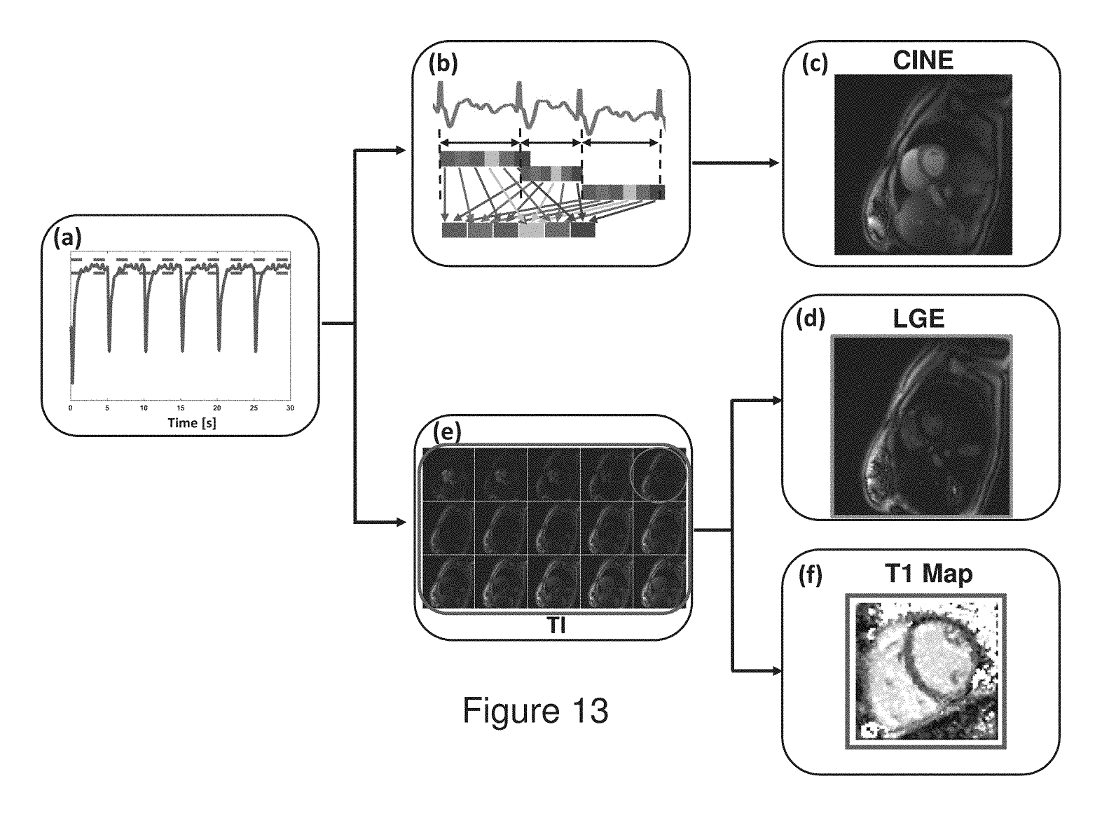

Other embodiments of the present disclosure relate to using, for example, magnetization preparation pulses played intermittently throughout the continuous acquisition to make the signal vary according to a property such as T1 relaxation or T2 relaxation. In such an embodiment, for example as shown in FIG. 11, a portion of the self-gated free breathing data can be used for cine images of cardiac function while other parts of the data can be used to make parametric maps of, for example, Native myocardial T1. By applying such a pulse sequence during the application of an injected contrast agent, myocardial perfusion can be measured. By applying this pulse sequence following injection of a contrast agent, a post contrast T1 map can be obtained or a late gadolinium enhanced image generated to assess myocardial scar or to derive extracellular volume maps (see FIG. 13). Embodiments of the present disclosure that simultaneously perform cine and T1 mapping may be referred to herein as CAT-SPARCS. CAT-SPARCS can be implemented in 2D, 2D SMS, and 3D imaging. In other embodiments, the magnetization can be made sensitive to other properties generating unique contrast-weighted images or maps in the same acquisition as for measurement of cine images.

Example Implementations and Corresponding Results

Various aspects of the present disclosure may be still more fully understood from the following description of example implementations and corresponding results and FIGS. 1-14. Some experimental data are presented herein for purposes of illustration and should not be construed as limiting the scope of the present disclosure in any way or excluding any alternative or additional embodiments.

Methods

Sampling Trajectory Design

To explore the interaction between the design of the spiral trajectory, the self-gating binning strategy and the reconstruction technique, 3 different slew-limited spiral trajectories were designed using the algorithm of Meyer et al. [30]. Uniform density spirals (UD), linear variable density spirals (VD), and dual-density spirals (DD) were evaluated. The dual density spiral design uses a Fermi-function shape for transition region as defined by [31]:

$$k ( n ) = {k}_{start} - \frac{{k}_{start} - {k}_{end}}{1 + {e}^{- τ ( n - {n}_{fs} )}} \qquad ( 1 )$$

Where kₛₜₐᵣₜ and k_{end} are the starting and ending density of the spiral, τ is the steepness that determines the sharpness of the transition area, and n_{f}_{ₛ} is the number of points in the fully sampled center of k-space. To achieve a fair comparison, 3 trajectories were designed to support the same spatial resolution, field of view (FOV), and the readout duration of spiral arm. The spiral trajectory was designed for a temporal resolution of around 40 ms which is the duration of 5 spiral interleaves each with a TR of 7.8 ms.

Cardiac and Respiratory Self-Gating Strategy

The automatic pipeline for generating cardiac and respiratory self-gating, and for performing the motion corrected reconstruction, is shown in FIG. 1. Self-gating cardiac signals were determined by gridding an 8×8 fully sampled central region of k-space for each spiral arm for all receiver coils (FIG. 1(a)), followed by low-pass temporal filtering to eliminate the high frequency component caused by the golden-angle sampling pattern. Next, PCA was performed on this data to derive 5 temporal-basis functions (FIG. 1(b)). To extract and determine the cardiac signal, a band-pass filter with a passband from 0.5 Hz to 2 Hz was applied on the 5 temporal basis functions. Then frequency spectrum analysis was used to find the cardiac motion related component by determining which basis function had the highest amplitude in the cardiac motion frequency range (FIG. 1(c)). Finally, the cardiac motion trigger was extracted by performing peak detection on the selected and filtered temporal basis function. To exclude potential artefactual peaks which are unrelated to the cardiac motion signal change, a threshold was set by taking mean of all the peaks and troughs (FIG. 1(d)(e)). To verify the accuracy of self-gating, the ECG signal was acquired during scanning to perform comparison by Bland-Altman analysis [32]. The respiratory gating signal can be obtained from the PCA data using a band-pass filter with a frequency range from 0.05 Hz to 0.5 Hz (FIG. 1(j)). This signal can be used for respiratory navigator binning. As expected the shape of this component is quite similar to the respiratory motion displacements derived from the linear registration (FIG. 1(h)(j)). As the motion-compensated reconstruction uses 2D linear shifts derived from registration techniques, the derived respiratory component was not used for further processing in this study. In this specific embodiment, all of the acquired data was utilized for image reconstruction. In other embodiments, a subset of the data may be rejected, such as from heart beats with different intervals in patients with arrhythmias, or to exclude data with significant respiratory motion. In other embodiments, the data may also be grouped based on the derived respiratory displacement or the respiratory signal described above for navigator binning.

Automatic Heart Detection

Using the detected cardiac triggers and a fixed cardiac phase number, which was calculated through dividing the mean R-R interval length by a fixed temporal resolution as 39 ms (5 spirals), a retrospective binning was performed. This results in 25-35 cardiac phases depending on the R-R interval. Then the same cardiac phase in different R-R intervals was combined to obtain a high-quality image series containing the cardiac motion (FIG. 1(f)). Next, an 80×80-pixel region of interest (ROI) containing the heart is automatically detected based on the fact that in cine images the heart region has the largest magnitude of change in signal intensity because of the cardiac motion. Thus, the ROI containing the heart can be automatically detected by finding the largest connected region of high standard deviation on a standard deviation map of signal intensity calculated from all of the frames of the dynamic dataset (FIG. 1(g)).

Respiratory Motion Correction

To correct the respiratory motion, it was assumed that for each R-R interval the respiratory position was constant. Using this assumption, the k-space data over each R-R interval was combined to create a static image for each heartbeat. Next, rigid registration is performed over the heart ROI to determine the in-plane displacements required to compensate for the bulk changes in the heart position resulting from respiratory motion (FIG. 1(h)). While breathing results in non-rigid motion of structures of the chest, the motion of a small rectangular ROI around the heart on a cardiac gated short-axis image can be reasonably approximated by in-plane rigid motion in the head-foot and anterior-posterior directions [33]. Rigid registration was performed by using mutual information as a metric to determine the rigid transformation from the source image to that of the target image [34]. In order to minimize respiratory drift, pairwise rigid registration of images was performed over an 11-frame window, which means the nth frame is registered from (n−5)^{th} to (n+5)^{th} frame. The obtained displacement information is used to derive the appropriate k-space linear phase shifts to register the heart. These linear phase shifts derived from each R-R interval combined image were applied to the acquired raw k-space data for each frame within that R-R interval as previously described [33]. While in this specific embodiment rigid registration is used for respiratory motion correction, in other embodiments, affine or non-rigid registration can be applied for correcting cardiac or respiratory motion.

Soft Retrospective Binning

After the raw k-space data were corrected for respiratory motion, the dataset was then retrospectively binned using a soft separation (FIG. 1(k)). The first fixed number of cardiac phases was calculated based on a fixed temporal resolution. When separating each R-R interval into different cardiac phases, instead of hard cutting each R-R interval separately, the end cardiac phase of the previous R-R interval may incorporate data from the first few spirals of the next R-R interval, if the number of frames in one R-R interval cannot be completely divided by the cardiac phase number. In other embodiments, other methods can be used to bin the data, such as prospective binning. In certain embodiments, implicit rather than explicit cardiac binning may be feasible.

Automatic Coil Selection

Several studies [35,36] have developed techniques for automatic coil selection to reduce streaking artifacts in radial acquisitions. In accordance with certain aspects of the present disclosure, a strategy was utilized to select coils based on the spiral artifacts within the automatically detected heart ROI (FIG. 1(i)). An artifact ratio was defined for the kth coil (rₖ) as shown in equation (2) where Re fₕₑₐᵣₜ is an aliasing-free multi-coil magnitude (reference) image that was reconstructed by using 100 continuous-acquired spirals, Imgₕₑₐᵣₜ indicates an under-sampled multi-coil magnitude image aliasing artifacts that was reconstructed using only 30 spirals, and const is a constant value calculated based on the energy difference of the reference and aliasing images.

$${r}_{k} = \frac{{‖ {Ref}_{heart} ( k ) - const \times {Img}_{heart} ( k ) ‖}_{2}}{{‖ {Ref}_{heart} ( k ) ‖}_{2}} , k ∈ [ 1 , N ] \qquad ( 2 )$$

To eliminate coils which predominantly contribute aliasing artifacts over the heart region, while still having an adequate number of coils for parallel imaging, ⅔ of the coils with the lowest artifact ratios were retained. As equation 2 represents the ratio of the artifact energy to the total energy in the image, a specific threshold to balance artifact energy versus signal to noise can be determined.

Image Reconstruction

Images in this study were reconstructed using low rank and sparse decomposition [29] (FIG. 1(l)). This method can reconstruct highly accelerated dynamic MM datasets by separating the background static-information from the dynamic information. In the reconstruction, the iterative SENSE algorithm [37] was adopted to enforce joint multi-coil low rank (L) and sparsity (S) simultaneously to exploit inter-coil correlations. Data compression in the low rank model was performed by truncating the singular value decomposition (SVD) representation of the dynamic image series, while in the S model it was done by discarding low-value coefficients in the temporal total variation domain. Coil sensitivity maps were computed from the temporal average of binned data using the adaptive coil combination technique [38]. Reconstruction parameters were chosen based on providing images with adequate reduction in aliasing artifacts with minimal visual temporal blurring of the endocardial border. The same set of parameters were used to reconstruct all datasets. The above-described embodiment demonstrates a specific implementation using low-rank and sparse image reconstruction. The SPARCS and CAT-SPARCS tests are not limited to this reconstruction, however, and other reconstruction techniques involving parallel imaging, compressed sensing, dictionary learning, model-based reconstruction, manifold learning, low-rank tensor reconstruction, or machine learning may alternatively be used with the disclosed self-gating free breathing reconstruction according to various embodiments.

Human Imaging

In one implementation, continuous spiral cine imaging was performed in 38 subjects. The subjects included 33 healthy volunteers and 5 patients undergoing clinical CMR studies. Written informed consent was obtained from all subjects, and imaging studies were performed under institutional review board (IRB) approved protocols.

Scanning was performed on a 3 T scanner (MAGNETOM Prisma, Siemens Healthineers, Erlangen, Germany) at the University of Virginia Medical Center. Image datasets were acquired using the standard body phased-array RF coil. Pulse sequence parameters included: FOV=320 mm, TR=7.8 ms, TE=lms, voxel size=1.25×1.25 mm², slice thickness=8 mm. A single spiral readout trajectory was rotated by the golden angle between subsequent TRs for data acquisition. Data was acquired for 16 seconds. The evaluation of the three trajectories was performed in 10 subjects. Images were reconstructed using the Non-Uniform Fast Fourier Transform (NUFFT) and L+S techniques. Two cardiologists, blinded to the acquisition trajectory, ranked the images produced with each of the trajectories, (1′-3^{rd}). The trajectory with the average best ranking was used for the rest of the cases. To evaluate quantification of LVEF, slices covering the whole heart were collected in 10 subjects. EF was determined by manual tracing of the endocardial borders by an experienced cardiologist. The calculated LVEF was compared to the standard clinical breath-hold ECG gated SSFP sequence.

For the other subjects, continuously acquired spiral data was obtained at a single short axis location. During the acquisition, ECG signal was also recorded. The R-R interval length from the ECG signal and extracted cardiac trigger were compared using Bland-Altman and linear regression plots. Images were reconstructed either from the whole 16 second data acquisition (2000 spirals) or using only 8 seconds worth of data (1000 spirals). The first 200 spirals in the acquired data were discarded to allow the signal to achieve steady state. Images were reconstructed using both NUFFT and L+S techniques.

Image quality for all datasets were assessed by 2 experienced cardiologists blinded to the reconstruction technique. Image quality was evaluated on a 5-point scale ranging from 1 (poor) to 5 (excellent). Comparison between the ranks/scores from the different techniques were compared using Friedman's test and Wilcoxon signed-rank tests for the comparisons between individual reconstruction techniques. The EF between the techniques was performed using a two-way ANOVA analysis with Tukey's Studentized Range test to correct for multiple comparisons. Statistical analysis was performed using SAS software 9.4 (SAS Institute Inc., Cary, N.C.).

Inversion Recovery Spiral Cine Imaging

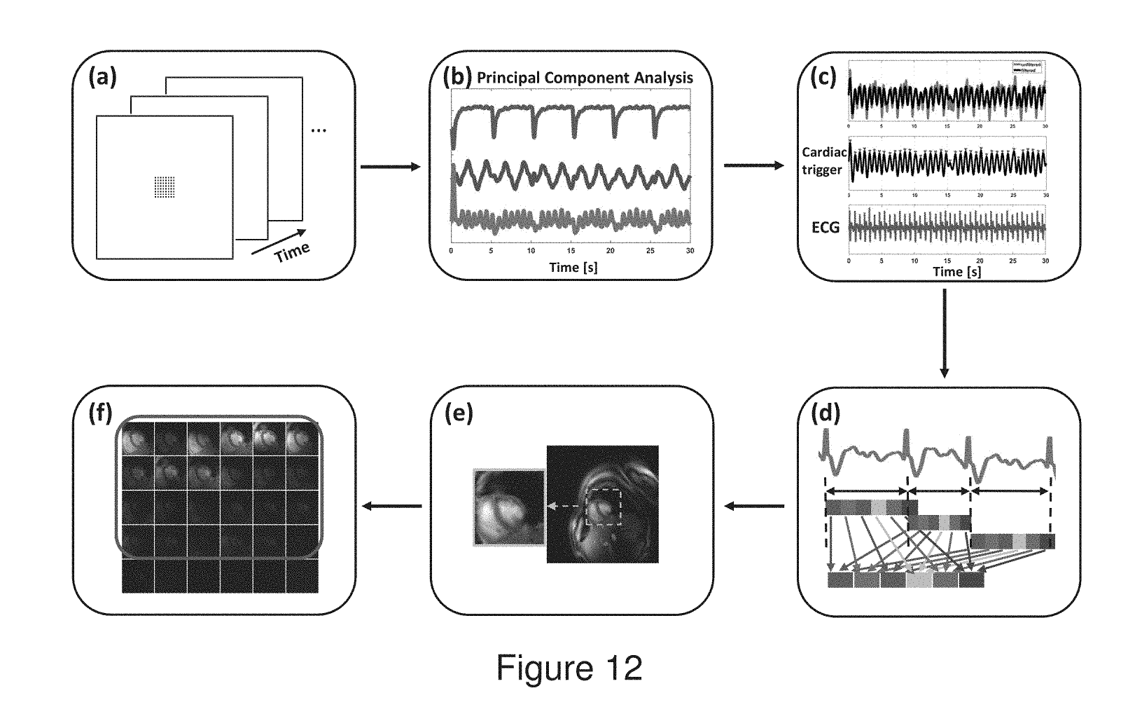

In one implementation, in order to derive LGE images and T1 maps, gradient echo data was acquired continuously for 30 seconds per slice using a pulse sequence consisting of a spiral trajectory rotated by the golden angle (GA). An adiabatic inversion pulse was applied every 5 seconds. Sequence parameters included: flip angle 15°, TR=7.5 ms, TE=1 ms, slice thickness=8 mm, in-plane resolution=1.5×1.5 mm. Self-gating cardiac signals, the respiratory pattern, and the signal recovery curve following inversion recovery (IR) were extracted by gridding an 8×8 central region of k-space of each spiral interleaf for all coils FIG. 12(a) followed by low-pass temporal filtering, principal component analysis (PCA) (FIG. 12(b)), and band-pass filtering of the derived temporal-basis functions with peak detection (FIG. 12(c)). The cardiac self-gating signal was used to retrospectively bin the data across the cardiac cycle (FIG. 12(d)), and automatic detection of the heart (FIG. 12(e)) and coil selection (FIG. 12(f)) were performed as described for SPARCS above.

With reference to FIG. 13, using the signal recovery curve derived from PCA, a threshold was chosen based on the steady state signal across the data set. The registered data was then separated into an LGE portion and a cine portion for image reconstruction. Cine images were reconstructed using low rank and sparsity (L+S) after performing retrospective cardiac binning with a reconstructed temporal resolution of 38 ms (5 GA spirals/frame). For the LGE image, a sliding window approach was used in the first few hundred milliseconds after the 2nd inversion pulse to determine the inversion time (TI) Then the same cardiac phase data at the chosen TI after each inversion pulse (except the 1st one) were combined to reconstruct an LGE image using SPIRiT with 150 ms temporal resolution. By binning the diastolic data during signal recovery, images can be created at multiple time points along the recovery curve needed to create a T1 map. The T1 map can be fit from the signal recovery based on analytic solutions to the Bloch equations. Alternatively the T1 recovery portion of the curve can be fit to a Bloch equation model which takes into account system imperfections such as inversion efficiency, B1 and B0 inhomogeneity, and flip angle slice profile.

Results

Cardiac and Respiratory Self-Gating

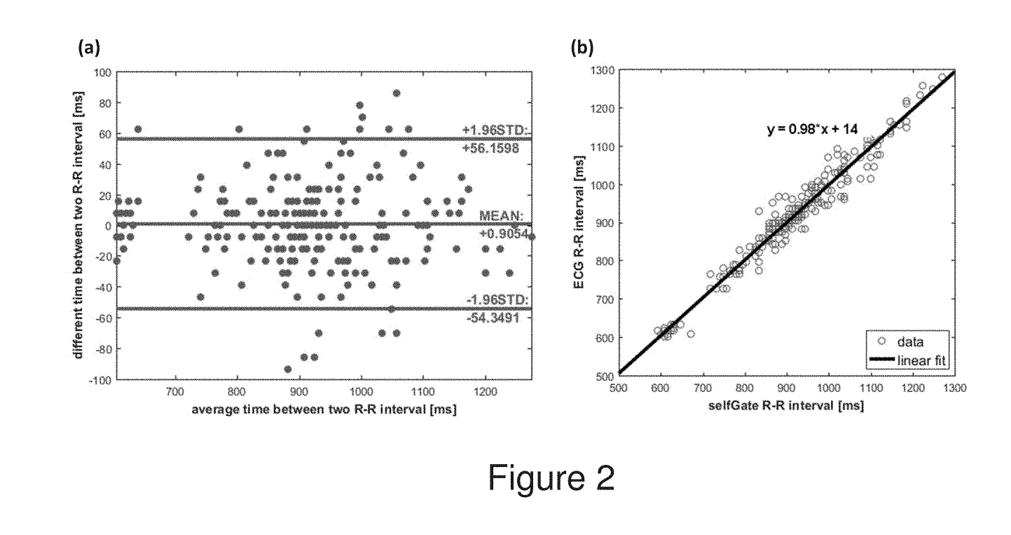

R-R interval length was compared between the ECG signal and the extracted cardiac trigger signal as shown in FIG. 2. Bland-Altman plots showed good agreement with no significant bias (p>0.05, paired sample t test) of lms, and linear regression of RR intervals demonstrated good correlation with R²=0.96.

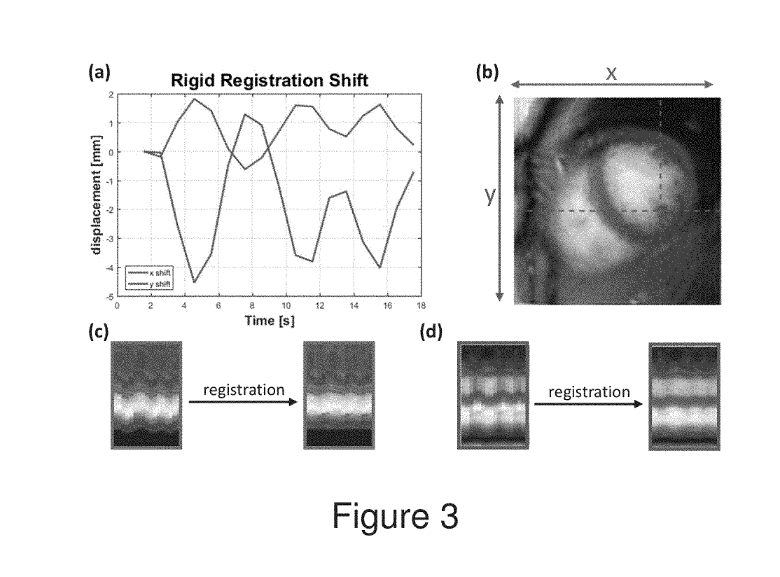

FIG. 3 shows the rigid registration performance from one subject. The x and y displacements are plotted in FIG. 3(a). The registration performance can be seen by comparing the x-t (FIG. 3(c)) and y-t (FIG. 3(d)) profiles before and after rigid registration. After registration, both x-t and y-t profiles are sharper and less corrupted by respiratory motion.

Automatic Coil Selection

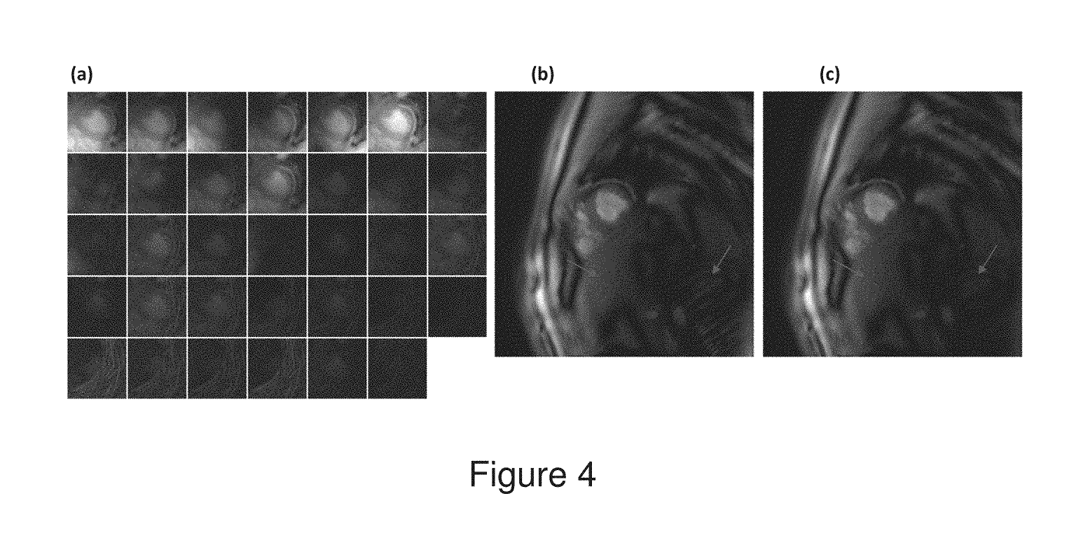

FIG. 4 demonstrates the automatic coil selection results. FIG. 4(a) shows the coil images in a region around the heart, and the coils are sorted from lowest to highest artifact energy from top left to bottom right. As expected, the coils that have a high SNR and low aliasing around the heart rank higher. FIG. 4(b)(c) shows the image result before and after automatic coil selection. The aliasing artifacts (red arrows) in the figure are significantly reduced by the coil selection process.

Evaluation of Cine Images

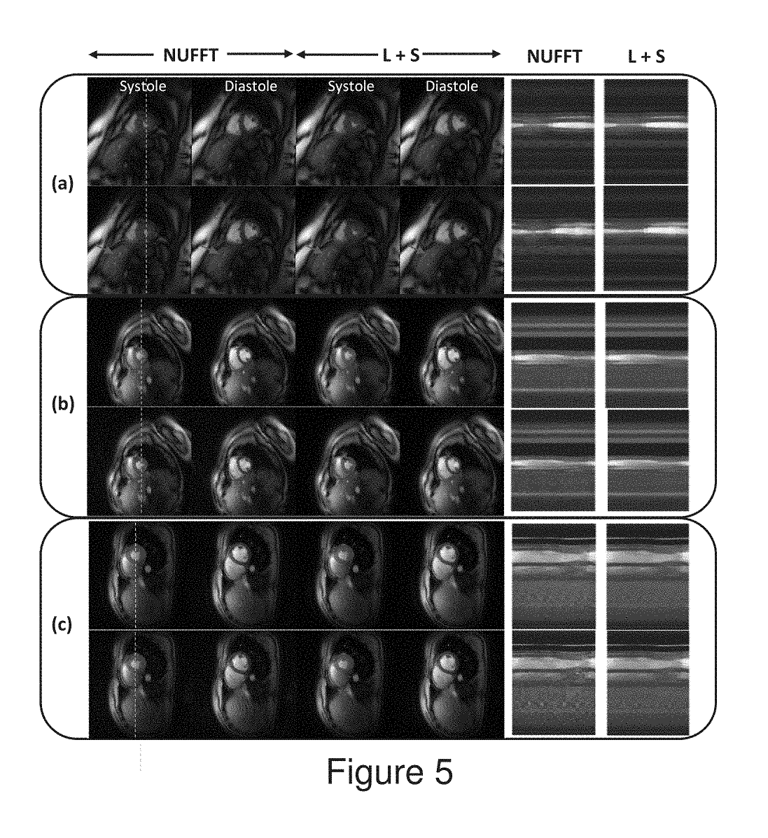

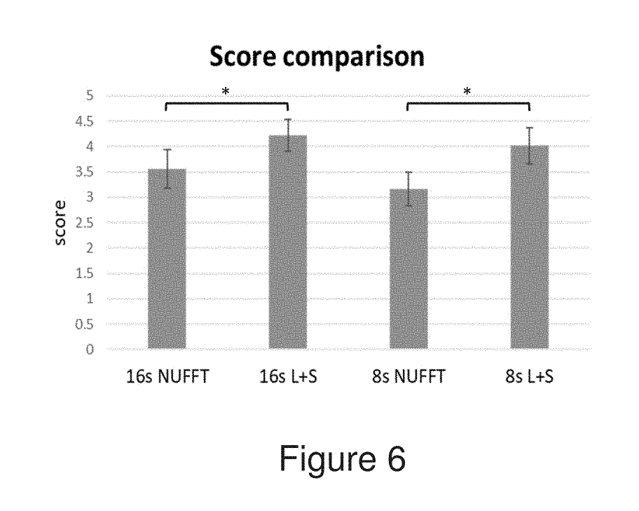

The first 4 columns in FIG. 5 shows cine images from three subjects at systole and diastole. FIG. 5(a-c) each represent different subjects. The first rows for all subjects correspond to reconstructed images using 16 s data while the second rows correspond to the ones using 8 s data. The first 2 columns show the direct gridding NUFFT results while the next 2 columns are the L+S reconstructed images. For column 1 and 3, the images are the end-systolic frames, and column 2 and 4 are the end-diastolic frames. The last 2 columns show the x-t profiles for NUFFT and L+S results, where the x positions refer to dashed lines in the images of the first column. For the three subjects, the images obtained using both 16 seconds and 8 seconds of data are shown. The NUFFT technique reconstructs each frame independently of the other frames and is thus free of any potential temporal blurring among frames, but it is less efficient at reducing spatial artifacts as compared to the L+S reconstruction. Alternatively, images may be constructed using a parallel imaging technique such as iterative SENSE, GRAPPA, SPIRiT, ESPIRIT, GROG or other methods which use coil sensitivity data and reconstruct data frame by frame without any temporal information. These methods may reduce aliasing artifacts as compared to NUFFT but may be more computationally complex. The L+S reconstruction significantly improves reconstruction quality for both 16 second and 8 second data, resulting in a reduction of residual aliasing artifacts without introducing significant visual temporal blurring. Scores (N=38) by 2 cardiologists are shown in FIG. 6. The mean (±standard deviation) image quality scores of the 4 types of reconstructions from left to right were 3.5±0.8, 4.2±0.6, 3.2±0.7 and 4.0±0.7. The L+S reconstruction was graded significantly higher than the NUFFT reconstruction for both the 8-second and 16-second datasets (p<0.001). There was no significant difference in image quality between the 8 second and 16 second L+S reconstructions.

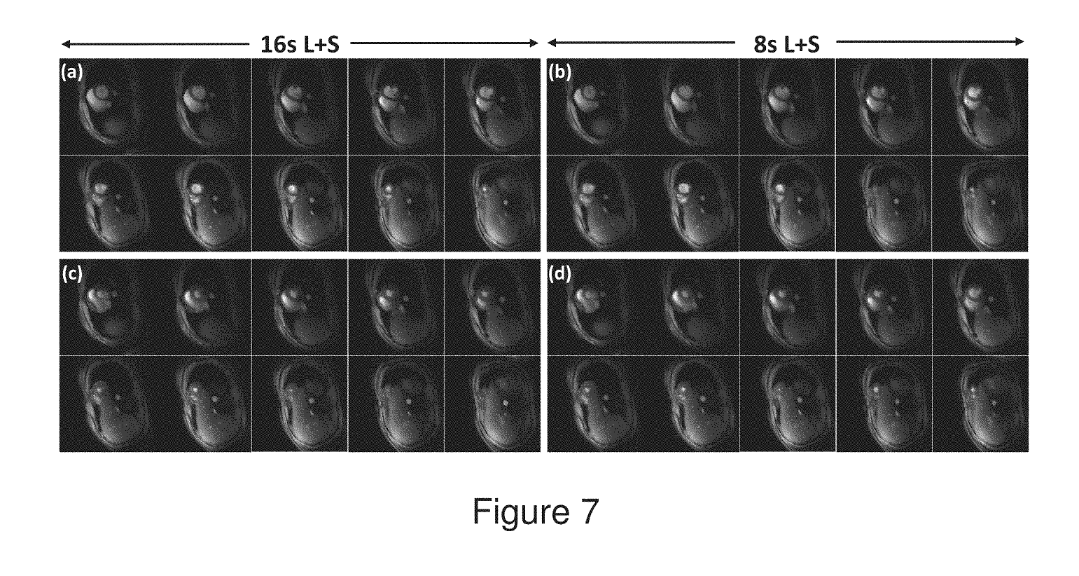

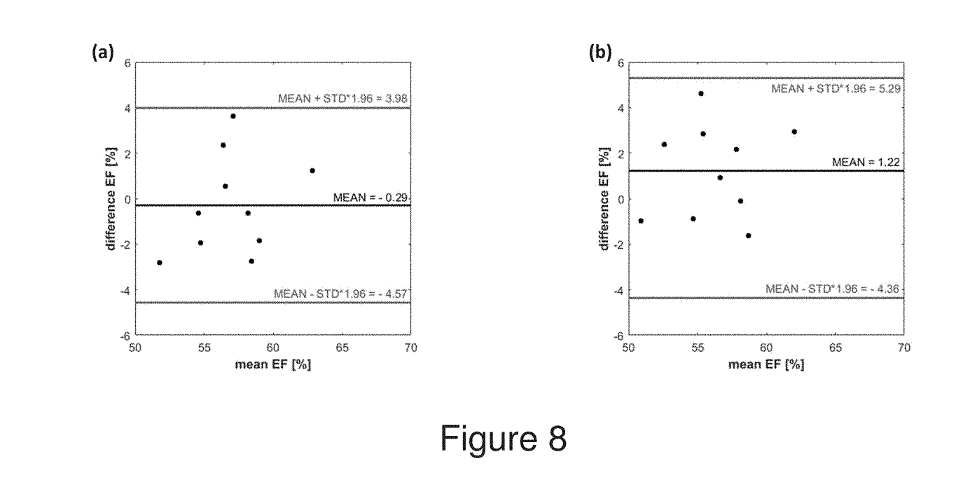

FIG. 7 shows the L+S reconstructed images from one subject with 10 slices covering the left ventricle. FIG. 7(a)(b) correspond to the diastolic frame and (c)(d) are a systolic frame. FIG. 7(a)(c) show the L+S reconstructed images using 16 s of data while (b)(d) used 8 s of data. Across all studies with whole ventricular data (N=10) the mean (±standard deviation) LVEF for 16 s NUFFT, 8 s NUFFT, 16 s L+S and 8 s L+S were 57.2±3.1, 56.0±3.3, 57.1±2.9 and 55.6±3.2 for the SPARCS technique as compared to 56.8±3.5 for the standard SSFP cine images. The Bland-Altman plot of EF between Cartesian SSFP images and 16 s L+S spiral images is shown in FIG. 8(a) while the Bland-Altman plot for the Cartesian SSFP images and 8 s L+S spiral images are shown in FIG. 8(b). ANOVA test showed no significant difference among the 5 groups (p>0.05), demonstrating the accuracy of calculating EF using the proposed SPARCS strategy.

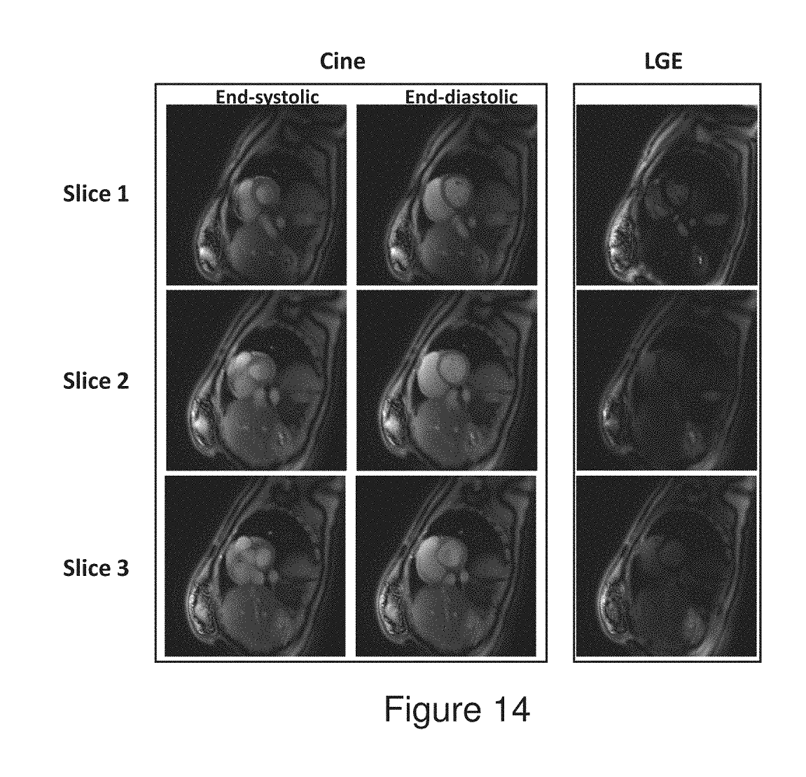

In one implementation of the present disclosure of an acquisition strategy used to simultaneously obtain cine and LGE images, after each inversion pulse, the signal intensity follows a T1* recovery curve. LGE images were obtained from data in the first few hundred milliseconds after each inversion pulse. Once the signal approaches steady state, cine images were generated. The derived cardiac trigger is consistent with the recorded ECG signal (FIG. 12(c)). FIG. 14 shows the cine image results from one subject at diastolic and systolic phases of three short-axis slices, as well as the LGE image at the corresponding slices. A T1 map can also be derived from the data which follows T1* recovery.

### DISCUSSION

In this work a free-breathing continuous-acquisition respiratory and cardiac self-gated golden angle spiral cine strategy (SPARCS) is disclosed. In accordance with one embodiment, a method for SPARCS used 8×8 center k-space from acquired data to derive a cardiac trigger without the need for ECG gating. Free breathing acquisition was also enabled by using a motion correction strategy during reconstruction. The method acquired data continuously and then retrospectively sorted the k-space data from each spiral into different cardiac phases based on the cardiac trigger derived from the self-gated signal. As the self-gated cardiac trigger and ECG signal performed similarly, the self-gating strategy provides a reasonable surrogate. To enable free breathing acquisition with 100% sampling efficiency, a rigid registration strategy was implemented to correct the motion caused by the respiratory motion between heart-beats. While currently there are more complex techniques for non-rigid registration [39,40], their performance is sensitive to image quality related factors, and their implementation for non-cartesian trajectories significantly increases reconstruction time and complexity. However, such techniques may be utilized in other embodiments of the proposed technology. Since most cardiac motion caused by breathing is in the head-foot and left-right directions, a rigid registration can be used. The inventors have previously demonstrated the robustness of this motion-correction strategy for myocardial perfusion imaging [33].

In terms of reconstruction for relatively low acceleration factors (2-3×), non-Cartesian SPIRiT [41] or non-Cartesian SENSE [42] performs well for spiral imaging. However, their performance is typically inadequate for higher acceleration rates. For more highly accelerated spirals techniques, compressed sensing approaches have been shown to improve reconstruction performance [31]. The L+S reconstruction method can provide a decomposition of low rank and sparsity components to separate background and dynamic components in an image. To be noted, the L component captures static and periodic motion in the background among cardiac phases, while the S component contains the dynamic cardiac motion information. Since the background has been suppressed, the S component has a sparser representation than the original matrix [29]. By exploiting the spatial and temporal correlation of the dynamic image series with iterative SENSE implementation, L+S method offers a quite efficient and robust reconstruction. In other embodiments of the present disclosure, alternative reconstruction techniques are utilized.

Although SSFP sequences are typically used for cine imaging at 1.5 T and 3 T, a gradient echo (GRE) strategy may have a few advantages for simplifying 3 T cine imaging. As the spiral trajectory has a long TR, there is time for inflow-enhancement of the LV blood pool resulting in a contrast which is similar to Cartesian SSFP imaging rather than that seen with short TR Cartesian GRE imaging. As the sequence is spoiled GRE-based rather than SSFP-based, a frequency scout, which is often needed for SSFP acquisition to avoid banding artifacts and out of plane flow artifacts, is not required. With 8 seconds of data acquisition per slice, the whole ventricle can be covered in about 90 seconds and with 16 seconds per slice the whole heart can be imaged in 3 minutes. The approach described herein can also be applied to spiral SSFP imaging at 1.5 T [14] or 3 T.

The idea of self-navigation was first pioneered by Larson et al. [23] for cardiac cine imaging using radial k-space sampling with SSFP sequence under breath hold condition, where the self-gated signal was extracted from echo peak magnitude, kymogram and 2D correlation. This idea was further explored by using a center k-space point [11], center k-space line [12] or processed center k-space data [25]. These cardiac self-gated methods typically use breath holds to avoid the complexity of separating cardiac motion and respiratory motion. Some studies also focused on free breathing imaging using navigator signals [18,19,43]. A respiratory and cardiac self-gated method by multi-echo 3D hybrid radial SSFP acquisition strategy was proposed by Liu et al., where coils were selected based on the smallest variance of either the RR intervals or respiratory positions for each individual coil. While in the present case, PCA is used to separate combinations of coils which correspond predominantly to the respiratory and cardiac signals. The optimal PCA basis functions for the cardiac and respiratory self-gating signal are determined by choosing the basis functions which have the highest amplitude in the cardiac or respiratory frequency ranges after band-pass filtering. Another study by Pang et al. [19] retrospectively binned the data into different cardiac and respiratory phases based on information extracted from self-gated projections and the different respiratory states were reconstructed to perform motion correction. This approach could have potential issues with subjects that have irregular breathing patterns resulting in some respiratory bins with not enough data to reconstruct a reasonable quality image to do motion correction between bins. Thus, the performance of binning the data into different respiratory bins might vary in individuals with different breathing patterns. In SPARCS according to certain embodiments of the present disclosure, respiratory motion was corrected for each R-R interval, which provides robustness to irregular breathing patterns. Compared with the previously proposed free breathing and/or cardiac self-gated strategies [20,24,25], SPARCS in accordance with certain embodiments of the present disclosure offers considerable improvements in spatial and temporal resolution with a short acquisition time.

Although the spiral based acquisitions may be more sensitive to off-resonance artifacts than Cartesian GRE, with short spiral readouts there is minimal spiral-induced blurring or dropout artifacts. Additionally, off-resonance correction can be applied to further improve off-resonance performance. GRE based acquisitions have lower CNR and SNR as compared to SSFP techniques. This is partially compensated for in the SPARCS technique by using a longer TR and higher flip angles. It has been demonstrated that SSFP-based spiral imaging is also feasible at 1.5 T, and it also can be performed at 3 T [44]. SPARCS may thus be implemented with either SSFP or GRE readouts.

### CONCLUSION

The specific configurations, choice of materials and the size and shape of various elements can be varied according to particular design specifications or constraints requiring a system or method constructed according to the principles of the present disclosure. Such changes are intended to be embraced within the scope of the present disclosure. The presently disclosed embodiments, therefore, are considered in all respects to be illustrative and not restrictive. The patentable scope of certain embodiments of the present disclosure is indicated by the appended claims, rather than the foregoing description, and all changes that come within the meaning and range of equivalents thereof are intended to be embraced therein.

### REFERENCE LIST

1. Keltner J R, Roos M S, Brakeman P R, Budinger T F. Magnetohydrodynamics of blood flow. Magn. Reson. Med. 1990; 16:139-49.
2. Polson M J R, Barker A T, Gardiner S. The effect of rapid rise-time magnetic fields on the ECG of the rat. Clin. Phys. Physiol. Meas. 1982; 3:231-4.
3. Shetty A N. Suppression of Radiofrequency Interference in Cardiac Gated MRI: A Simple Design. Magn. Reson. Med. 1988; 8:84-8.
4. Damji A A, Snyder R E, Ellinger D C, Witkowski F X, Allen P S. RF interference suppression in a cardiac synchronization system operating in a high magnetic field NMR imaging system. Magn. Reson. Imaging. 1988; 6:637-40.
5. Firmin D, Keegan J. Navigator echoes in cardiac magnetic resonance. J. Cardiovasc. Magn. Reson. 2001; 3:183-93.
6. Jung B, Zaitsev M, Hennig J, Markl M. Navigator gated high temporal resolution tissue phase mapping of myocardial motion. Magn. Reson. Med. 2006; 55:937-42.
7. Peters D C, Nezafat R, Eggers H, Stehning C, Manning W J. 2D free-breathing dual navigator-gated cardiac function validated against the 2D breath-hold acquisition. J. Magn. Reson. Imaging. 2008; 28:773-7.
8. Abd-Elmoniem K Z, Obele C C, Sibley C T, Matta J R, Pettigrew R I, Gharib A M. Free Breathing Single Navigator Gated Cine Cardiac Magnetic Resonance at 3 Tesla: Feasibility Study in Patients. J Comput Assist Tomogr. 2011; 35:382-6.
9. Moghari M H, Barthur A, Amaral M E, Geva T, Powell A J. Free-breathing whole-heart 3D cine magnetic resonance imaging with prospective respiratory motion compensation. Magn. Reson. Med. [Internet]. 2017; 189:181-9. Available from: http://doi.wiley.com/10.1002/mrm.27021
10. Crowe M E, Larson A C, Zhang Q, Carr J, White R D, Li D, et al. Automated rectilinear self-gated cardiac cine imaging. Magn. Reson. Med. 2004; 52:782-8.
11. Buehrer M, Curcic J, Boesiger P, Kozerke S. Prospective self-gating for simultaneous compensation of cardiac and respiratory motion. Magn. Reson. Med. 2008; 60:683-90.
12. Uribe S, Muthurangu V, Boubertakh R, Schaeffter T, Razavi R, Hill D L G, et al. Whole-heart cine MRI using real-time respiratory self-gating. Magn. Reson. Med. 2007; 57:606-13.
13. Uribe S, Beerbaum P, Sorensen T S, Rasmusson A, Razavi R, Schaeffter T. Four-dimensional (4D) flow of the whole heart and great vessels using real-time respiratory self-gating. Magn. Reson. Med. 2009; 62:984-92.
14. Feng X, Salerno M, Kramer C M, Meyer C H. Non-Cartesian balanced steady-state free precession pulse sequences for real-time cardiac MRI. Magn. Reson. Med. 2016; 75:1546-55.
15. Stehning C, B??rnert P, Nehrke K, Eggers H, Stuber M. Free-breathing whole-heart coronary MRA with 3D radial SSFP and self-navigated image reconstruction. Magn. Reson. Med. 2005; 54:476-80.
16. Lai P, Bi X, Jerecic R, Li D. A Dual-Projection Respiratory Self-Gating Technique for Whole-Heart Coronary MRA. Magn. Reson. Med. 2009; 62:731-8.
17. Lai P, Bi X, Jerecic R, Li D. A respiratory self-gating technique with 3D-translation compensation for free-breathing whole-heart coronary MRA. Magn. Reson. Med. 2009; 62:731-8.
18. Usman M, Atkinson D, Odille F, Kolbitsch C, Vaillant G, Schaeffter T, et al. Motion corrected compressed sensing for free-breathing dynamic cardiac MRI. Magn. Reson. Med. 2013; 70:504-16.
19. Pang J, Bhat H, Sharif B, Fan Z, Thomson L E J, Labounty T, et al. Whole-heart coronary MRA with 100% respiratory gating efficiency: Self-navigated three-dimensional retrospective image-based motion correction (TRIM). Magn. Reson. Med. 2014; 71:67-74.
20. Usman M, Ruij sink B, Nazir M S, Cruz G, Prieto C. Free breathing whole-heart 3D CINE MRI with self-gated Cartesian trajectory. Magn. Reson. Imaging. The Authors; 2017; 38:129-37.
21. Liu J, Spincemaille P, Codella N C F, Nguyen T D, Prince M R, Wang Y. Respiratory and cardiac self-gated free-breathing cardiac CINE imaging with multiecho 3D hybrid radial SSFP acquisition. Magn. Reson. Med. 2010; 63:1230-7.
22. Pang J, Sharif B, Fan Z, Bi X, Arsanjani R, Berman D S, et al. ECG and navigator-free four-dimensional whole-heart coronary MRA for simultaneous visualization of cardiac anatomy and function. Magn. Reson. Med. 2014; 72:1208-17.
23. Larson A C, White R D, Laub G, McVeigh E R, Li D, Simonetti O P. Self-Gated Cardiac Cine MRI. Magn. Reson. Med. 2004; 51:93-102.
24. Han F, Rapacchi S, Hu P. Prospective cardiac motion self-gating. Quant Imaging Med Surg. 2017; 2:215-26.
25. Bonanno G, Hays A G, Weiss R G, Schar M. Self-gated golden angle spiral cine MRI for coronary endothelial function assessment. Magn. Reson. Med. 2017; 00:1-11.
26. Odille F, Uribe S, Batchelor P G, Prieto C, Schaeffter T, Atkinson D. Model-based reconstruction for cardiac cine MM without ECG or breath holding. Magn. Reson. Med. 2010; 63:1247-57.
27. Brau A C S, Brittain J H. Generalized self-navigated motion detection technique: Preliminary investigation in abdominal imaging. Magn. Reson. Med. 2006; 55:263-70.
28. Larson A C, Kellman P, Arai A, Hirsch G A, McVeigh E, Li D, et al. Preliminary investigation of respiratory self-gating for free-breathing segmented cine MRI. Magn. Reson. Med. 2005; 53:159-68.
29. Otazo R, Candés E, Sodickson D K. Low-rank plus sparse matrix decomposition for accelerated dynamic MRI with separation of background and dynamic components. Magn. Reson. Med. 2015; 73:1125-36.
30. Meyer C, Hu B, Nishimura D, Macovski A. Fast Coronary Artery Imaging. Magn. Reson. Imaging. 1992; 28:202-13.
31. Yang Y, Kramer C M, Shaw P W, Meyer C H, Salerno M. First-pass myocardial perfusion imaging with whole-heart coverage using L1-SPIRiT accelerated variable density spiral trajectories. Magn. Reson. Med. 2016; 76:1375-87.
32. Martin Bland J, Altman D. Statistical Methods for Assessing Agreement Between Two Methods of Clinical Measurement. Lancet. 1986; 327:307-10.
33. Zhou R, Huang W, Yang Y, Chen X, Weller D S, Kramer C M, et al. Simple motion correction strategy reduces respiratory-induced motion artifacts for k-t accelerated and compressed-sensing cardiovascular magnetic resonance perfusion imaging. J. Cardiovasc. Magn. Reson. Journal of Cardiovascular Magnetic Resonance; 2018; 20:1-13.
34. Smriti R, Stredney D, Schmalbrock P, Clymer B D. Image Registration Using Rigid Registration and Maximization of Mutual Information. MMVR13 (The 13th Annu. Med. Meets Virtual Real. Conf. Long Beach, Calif., USA; 2005.
35. Xue Y, Yu J, Kang H S, Englander S, Rosen M A, Song H K. Automatic coil selection for streak artifact reduction in radial MM. Magn. Reson. Med. 2012; 67:470-6.
36. Feng L, Huang C, Shanbhogue K, Sodickson D K, Chandarana H, Otazo R. RACER-GRASP: Respiratory-weighted, aortic contrast enhancement-guided and coil-unstreaking golden-angle radial sparse MRI. Magn. Reson. Med. 2017; 00.
37. Klaas P. Pruessmann, Advances in Sensitivity Encoding With Arbitrary\nk-Space Trajectories. 2001; 651:638-51. Available from: http://www.mr-tip.com/servl.php?type=co
38. Walsh D O, Gmitro A F, Marcellin M W. Adaptive reconstruction of phased array MR imagery. Magn. Reson. Med. 2000; 43:682-90.
39. Rao A, Sanchez-Ortiz G I, Chandrashekara R, Lorenzo-Valdes M, Mohiaddin R, Rueckert D. Comparison of cardiac motion across subjects using non-rigid registration. Med. Image Comput. Comput. Interv. —MICCAI 2002. 2002; 2488:722-9.
40. Chen X, Salerno M, Yang Y, Epstein F H. Motion-compensated compressed sensing for dynamic contrast-enhanced MRI using regional spatiotemporal sparsity and region tracking: Block low-rank sparsity with motion-guidance (BLOSM). Magn. Reson. Med. 2014; 72:1028-38.
41. Lustig M, Pauly J M. SPIRiT: Iterative self-consistent parallel imaging reconstruction from arbitrary k-space. Magn. Reson. Med. 2010; 64:457-71.
42. Pruessmann K P, Weiger M, Scheidegger M B, Boesiger P. SENSE: Sensitivity encoding for fast MRI. Magn. Reson. Med. 1999; 42:952-62.
43. Harrison A, Adluru G, Damal K, Shaaban A M, Wilson B, Kim D, et al. Rapid ungated myocardial perfusion cardiovascular magnetic resonance: Preliminary diagnostic accuracy. J. Cardiovasc. Magn. Reson. 2013; 15:1-10.
44. Barkauskas K J, Rajiah P, Ashwath R, Hamilton J I, Chen Y, Ma D, et al. Quantification of left ventricular functional parameter values using 3D spiral bSSFP and. J. Cardiovasc. Magn. Reson. 2014; 1-13.
45. Yang Y, Meyer C M, Epstein F H, Kramer C M, Salerno M. “Whole Heart Spiral Simultaneous Multi-Slice First-Pass Myocardial Perfusion Imaging.” Magnetic Reson Med 2018.

What is claimed is:

1. A method for free-breathing cine imaging of an area of interest of a subject, comprising:
acquiring, during free breathing of the subject, magnetic resonance imaging data corresponding to an area of interest of a subject that comprises the heart, wherein the acquiring comprises applying a pulse sequence with a spiral trajectory;
performing cardiac self-gating using a self-gating signal extracted from a central region of k-space;
performing respiratory motion correction to compensate for changes in the heart position during respiratory motion, wherein the motion correction comprises rigid or non-rigid registration to determine corrective displacements; and
performing image reconstruction to produce cine images of the area of interest over a plurality of heart-beats.

2. The method of claim 1, wherein the pulse sequence is a gradient echo spiral pulse sequence with a spiral trajectory rotated by the golden angle in time.

3. The method of claim 2, wherein performing the cardiac self-gating comprises extracting the self-gating signal from a fully sampled region of k-space.

4. The method of claim 3, wherein extracting the self-gating signal from the fully sampled central region of k-space comprises principal component analysis (PCA).

5. The method of claim 1, wherein the pulse sequence uses a variable density spiral with a fully sampled center.

6. The method of claim 1, wherein the pulse sequence uses a uniform density spiral.

7. The method of claim 1, wherein the pulse sequence uses a dual density spiral.

8. The method of claim 1, wherein the image reconstruction comprises decomposition of low rank and sparsity components to separate background and dynamic components.

9. The method of claim 1, wherein the pulse sequence is a steady-state free precession pulse sequence.

10. The method of claim 1, wherein the pulse sequence has a spiral trajectory wherein the spirals are rotated in time by an angle differing from the golden angle.

11. The method of claim 1, wherein the image reconstruction is performed using at least one of parallel imaging, compressed sensing, dictionary learning, model based reconstruction, low rank tensor reconstruction, manifold learning, or machine learning.

12. The method of claim 1, wherein the area of interest comprises the whole heart of the subject.

13. The method of claim 1, wherein the area of interest is restricted to a region around the heart using outer-volume suppression or inner volume selection.

14. The method of claim 1, wherein acquiring the magnetic resonance imaging data comprises performing simultaneous multi-slice imaging.

15. The method of claim 1, wherein the pulse sequence comprises a stack-of-spirals trajectory used to cover a 3d volume.

16. The method of claim 1, wherein the pulse sequence uses spirals with a slice selection gradient played out during readout to cover a 3d volume with cones.

17. The method of claim 1, wherein the pulse sequence is applied during or after injection of a contrast agent into the subject.

18. The method of claim 1, wherein a T1, T2, or other magnetization preparation are performed one or more times during the acquisition to cause a signal intensity variation.

19. The method of claim 1, comprising generating, from part of the acquired magnetic resonance imaging data, a static image depicting myocardial scarring.

20. The method of claim 1, comprising generating, from part of the acquired magnetic resonance imaging data, a parametric map of T1 or T2 relaxation times.

21. The method of claim 1, wherein navigation is performed using a navigator signal played out during continuous acquisition using a rectilinear linear, spiral, cone, or other trajectory.

22. A system for free-breathing cine imaging of an area of interest of a subject, comprising:
a data acquisition device configured to acquire, during free breathing of the subject, magnetic resonance imaging data corresponding to an area of interest of a subject that comprises the heart, wherein the acquiring comprises applying a pulse sequence with a spiral trajectory; and
one or more processors coupled to the data acquisition device and configured to cause the system to perform functions including:
performing cardiac self-gating using a self-gating signal extracted from a central region of k-space;
performing respiratory motion correction to compensate for changes in the heart position during respiratory motion, wherein the motion correction comprises rigid or non-rigid registration to determine corrective displacements; and
performing image reconstruction to produce cine images of the area of interest over a plurality of heart-beats.

23. A non-transitory computer-readable medium having stored instructions that, when executed by one or more processors, cause a magnetic resonance imaging system to perform functions that comprise:
acquiring, during free breathing of the subject, magnetic resonance imaging data corresponding to an area of interest of a subject that comprises the heart, wherein the acquiring comprises applying a pulse sequence with a spiral trajectory;
performing cardiac self-gating using a self-gating signal extracted from a central region of k-space;
performing respiratory motion correction to compensate for changes in the heart position during respiratory motion, wherein the motion correction comprises rigid or non-rigid registration to determine corrective displacements; and
performing image reconstruction to produce cine images of the area of interest over a plurality of heart-beats.
